(19) United States
(12) Patent Application Publication
(10) Pub. No.: US 2009/0192209 A1
(43) Pub. Date: Jul. 30, 2009

(54) Extended treatment of tumors through vessel occlusion with light activated drugs

(75) Inventors: Paula A. Mahoney, Woodinville, WA (US); Hongxia Xu, Bellevue, WA (US); Michael Alan Krouse, Seattle, WA (US); Yan Liang, Bellevue, WA (US); Myron Jay Winship, North Bend, WA (US)

Correspondence Address: SEED INTELLECTUAL PROPERTY LAW GROUP PLLC, 701 FIFTH AVE, SUITE 5400, SEATTLE, WA, 98104, US

(73) Assignee: Light Sciences Oncology, Inc., Bellevue, WA (US)

(21) Appl. No.: 11/663,695

(22) PCT Filed: Jan. 7, 2005
(86) PCT No.: PCT/US05/00636

§ 371 Date: Apr. 1, 2008

Related U.S. Application Data
(60) Provisional application No. 60/612,929, filed on Sep. 24, 2004.

(51) Int. Cl. A61K 31/40

(52) U.S. Cl. 514/410

(57) ABSTRACT

Methods of treating diseases of a subject through selective occlusion of blood vessels. The methods include identifying and/or mapping the blood vessels supplying nutrients to the diseased or unwanted tissue, delivering an appropriate dose of an occlusion-forming photosensitizer to the subject, and exciting the photosensitizer with sufficient light irradiation in the vicinity of the blood vessels to cause vessel occlusion. The vessel occlusion cuts off nutrient supply to the diseased or unwanted tissue resulting in cell death of that tissue and any other tissue served by the occluded vessels.

### BACKGROUND OF THE INVENTION

1. Field of the Invention

The invention relates to the treatment of tumors, using a light activated (photoactive) drug, and a light source for activation of the drug.

2. Description of the Related Art

In photodynamic therapy (PDT), classes of photoreactive compounds, also known as “photosensitizers”, are excited with specific illumination wavelengths in order to treat diseased or undesirable tissue. Photosensitizers may be activated by at least one wavelength of light (“the excitation wavelength”) and are used in combination with light sources, often provided as laser light, to treat targeted tissue in a variety of eye, cardiac, oncological and other disease conditions.

Two important and related components of a photo reactive treatment system are the photosensitizer and the apparatus for supplying an excitation light to targeted tissue. The light source must meet various requirements including light wavelength, intensity, duration, and shape. Inappropriate illumination, such as misdirected or misshaped illumination, or excessive intensity, could cause photosensitizers to injure normal healthy tissue. Less light dose than necessary could result in ineffective treatment requiring further subsequent action. In the case of the photosensitizer, it must be effective, non-toxic, a non-irritant or at least well tolerated.

Tumors have been treated directly using photodynamic therapy. In general, the subject receives a dose of a photosensitizer, and then the photosensitizer in and around the tumor is activated by light. The light may be supplied from an external source and beamed through the skin of the subject or a device may be inserted into the subject and internally disposed to provide light. The light may be laser light or non-laser light. Because each light source has a limited effective illumination zone within which it can effectively excite the photosensitizer, treatment of larger tumors often necessitates use of multiple light sources directed at surface areas of the tumor to treat the large tumor effectively. Similarly, tumors dispersed within a body organ such as a liver may require multiple light sources, often several directed at each tumor, for effective treatment. The use of multiple light sources, especially if implanted into the subject, poses several issues that could be avoided if fewer sources, or only one light source, could be used.

### SUMMARY

The invention provides methods of treating a subject having a medical condition that is susceptible to treatment through occlusion of blood vessels. The invention provides methods for indirectly treating diseased tissue through occlusion of vasculature that supplies nutrients to the diseased tissue and some healthy tissue in the vicinity of the diseased tissue. While this inevitably leads to cell death of some nearby healthy tissue served by the same vessels, this healthy tissue might also be removed or destroyed in alternative surgical and other treatment process to ensure the permanent removal of the diseased tissue, and any possible diseased cells dispersed within healthy nearby tissue.

In one aspect, the invention provides methods of treating tumors and other abnormal growths through identifying and locating one or more major blood vessels supplying these with nutrients. The subject receives a therapeutically effective dose of a photosensitizer and in one embodiment a device, such as a light bar, is brought within effective illumination range of the vessels so that light from the bar excites the photosensitizer and causes occlusion of the vessels. The vessel occlusion results in a cut off of nutrients to the diseased or unwanted tissue, and ultimately to tissue cell death. In other instances, other light sources may be used, for example, laser light may be applied from a device outside the body and directed into the body.

Contrary to the prior art, the invention does not rely for its treatment effects on the direct photo-ablation of any diseased tissue, nor does it rely on the occlusion of only abnormal new vessels (“neovasculature”) although both such activities may also take place to some extent as incidental to the methods of the invention. Rather, the invention relies upon the occlusion of ordinary nutrient-supplying blood vessels that supply the diseased tissue as well as commonly-supplied surrounding healthy tissue. Because of this, the invention affects an extended area (i.e. has an extended therapeutic range) beyond the effective range of penetration of the light dose that excites the photosensitizer. The recognition that nutrient-supplying vessel occlusion is a mechanism whereby an effective treatment can be carried out is a significant advance in the technology and permits a different approach to treatment than direct treatment of diseased or unwanted tissue. The mapping of blood vessel location is therefore important so that light to excite a photosensitizer may be delivered more precisely to the vessels supplying nutrients rather than to the tumors or unwanted tissue.

In accordance with the invention, since blood supply to multiple tumors, dispersed within a large volume within an organ, may stem from one or more main supply blood vessels, the positioning of a light source to activate the photosensitizer is dictated by the location of these main blood vessels rather than the multiple tumor locations. Appropriate light dose delivery to occlude the major vessels in a single area at or in the vicinity of the main blood vessel may then effectively treat a large area of an organ affected by dispersed neoplasia.

The procedures of the invention are potentially less invasive in those cases where implantable light sources are used: fewer light emitting devices have to be inserted into the subject's affected organ than might be the case if each tumor dispersed within the organ had to be treated separately. Further, because of the expanded zone within which cell death occurs, any potential diseased cells dispersed within the immediate surrounding tissue served by the same blood vessels are also destroyed. Accordingly, the potential for subsequent proliferation of diseased cells is reduced.

### BRIEF DESCRIPTION OF THE DRAWINGS

The following drawings are illustrative of aspects of the invention and do not limit the invention as described herein. The drawings are not to scale and are intended only for explanatory purposes.

FIG. 1 shows a schematic of a tumor that is supplied by a single main blood vessel; and

FIG. 2 shows a schematic of a group of tumors supplied by a network of blood vessels, of which one is the primary blood vessel.

### DETAILED DESCRIPTION OF THE PREFERRED EMBODIMENTS

In the past, photodynamic therapy has been carried out by tumor ablation using externally applied laser light or percutaneously implanted light-emitting diodes (LEDs). In clinical tests, PDT with Talaporfin Sodium as photosensitizer has shown objective response rates of 33-50% in targeted tumors, including liver metastases arising from various primaries (Cancer 2003; 98:1767-71). It was believed that treatment of larger tumors required placement of multiple light sources around the tumor to illuminate and photo-ablate or otherwise directly destroy the cells of the tumor. Likewise, where there was a plurality of tumors, it was considered that several light sources were required to ensure treatment of each tumor.

It has now been observed that the blood vessels supplying nutrients to the tumors or unwanted tissue can be occluded and the volume of tissue supplied by these vessels will be destroyed. The size and shape of the volume of tissue destroyed constitutes an “extended “PDT-effect zone”. This PDT-effect zone is referred to as “extended” because it extends beyond the immediate surroundings of the PDT procedure zone (where photosensitizer was present and light applied) to include those more remote regions that the occluded vessels formerly supplied with blood.

In accordance with this finding, the invention provides a methodology of treating a single tumor or multiple tumors by first identifying the location of major blood vessels supplying nutrient to these (and surrounding healthy tissue) and then selectively and strategically treating these with PDT to cause occlusion. Note that these major vessels will also supply healthy tissue in the area of the tumors (“surrounding healthy tissue”) with blood and so inevitably this commonly fed tissue will also be adversely affected. However, this is not a serious disadvantage since such tissue is often removed or destroyed if alternative surgical or other procedures are used to remove or treat the tumors, to reduce the likelihood of subsequent new tumor growth.

In general, the invention first identifies the “target area” of the organ affected with neoplasia. It then finds the blood vessel(s) supplying the target area and identifies the major blood vessels. Preferably, there should be only one such vessel, but often there is a network of vessels. Through mapping of the vessels in the area, main vessels supplying the tumor affected area can be identified for treatment. Some of these vessels may supply blood directly to tumors while others may supply blood indirectly, via other intervening and connected vessels with which they are in fluid communication. The invention then provides the subject with an appropriate dose of a blood vessel-occluding photosensitizer. Thereafter, an appropriate wavelength light dose is applied to these major vessels, either through an implanted device or from externally. As a result, the vessels are occluded and the entire target area supplied by these vessels is effectively treated in that cell death occurs in all tissue formerly supplied by the vessels.

In any particular procedure, the PDT practitioner must select a photosensitizer dose to match the size of vessel to be occluded, taking into account such factors as the organ of the body in which the tumor or unwanted tissue is located, how rapidly it is cleared from the organ under consideration, and the vessel-occlusion properties of the particular photosensitizer. In general, a person of skill in the art must ensure a sufficient concentration of photosensitizer is present in the immediate surroundings of the blood vessel or vessels to be occluded, and that PDT is carried to completely occlude these.

This invention can be used with any PDT regime that uses a photosensitizer that causes blood vessel occlusion to (a) entirely eliminate a large tumor and surrounding tissue that may be remote from the immediate PDT-effect zone (i.e. that extend to beyond the effective reach of the light dose), or to (b) completely eliminate multiple tumors and their surrounding tissue that may be remote from the immediate PDT-effect zone; as long as the remote tissue is served by the PDT-occluded blood vessels.

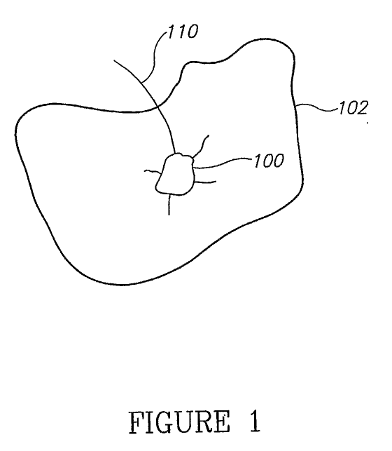

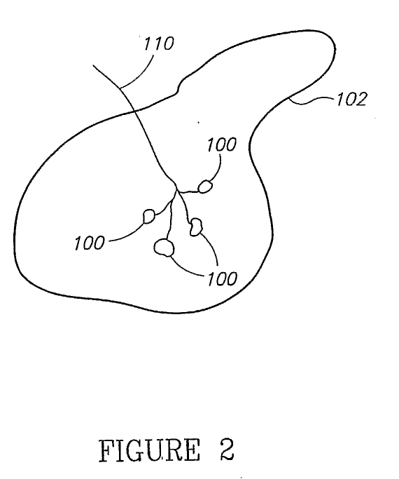

The invention provides significant benefits in treatment of tumors and unwanted tissue in any body organ. Embodiments of the invention are shown schematically in FIGS. 1 and 2.

In FIG. 1, the tumor 100 is located within an organ 102, such as the liver, spleen, etc., and a vascular mapping procedure has identified a main blood vessel 110 supplying oxygen and nutrients to tumor 100. Mapping may be carried out by any of a number of techniques known in the art. For example, intravascular dye angiography, color flow Doppler, CT-enhanced vascular imaging (computer assisted tomography), MRI scans, and radio-nucleotide imaging. According to the invention, the subject receives an appropriate dose of a vessel-occluding photosensitizer. Then, after photosensitizer is in the vicinity of the vessel, a device with an appropriate light source to excite the photosensitizer is inserted into the body (or in some cases light may be applied through the skin from outside the body) to a region in close proximity to the blood vessel. Irradiation with light proceeds for a period of time that is dependent upon several factors, and especially upon the particular photosensitizer being used, its dose and the size of the vessels to be occluded. After an appropriate period of irradiation, the irradiation is discontinued and the device is withdrawn. The PDT results in occlusion of the blood vessel that in turn leads to starvation of the tumor (and surrounding healthy, but perhaps contaminated, tissue served by the vessel) over a period of time, and ultimately death of the tumor cells (and some surrounding tissue cells).

In FIG. 2, an organ 102 has multiple tumors 100 dispersed within a target zone, but each are linked ultimately via a branched network of vessels to a common vessel 110. As above, occlusion of this vessel 110 via PDT results in effective treatment of the entire area that encapsulates tumors 100, resulting in cell death of tumor cells as well as surrounding healthy cells in that area. Thus treatment with a single light source can according to the invention treat a plurality of dispersed tumors, as long as a few major vessels that are common and supply nutrients to all are treated to occlude them.

The invention is broadly applicable wherever occlusion of blood vessels, supplying nutrients to diseased and/or unwanted tissue, is a useful method of treatment. Some non-limiting examples of potential application include treatment of large tumors, areas with multiple tumors at early or advanced stage, tumors in which surgical resection carries a high risk due to location near to a critical structure. Beyond oncology, this approach would be useful in treating other diseases associated with neovasculature, for example in the cardiovascular or ophthalmology areas, and the like. This approach may also find use in elimination of localized tissue such as fat cell deposits, that have identifiable blood supply vessels that can be strategically occluded without significant harm to the subject.

The following example illustrates aspects of the invention, and does not limit the scope of the invention as described herein and here below claimed.

### EXAMPLE

A study was carried out to explore whether placement of a single light source in proximity to an identified tumor-feeding vessel could result in effective treatment of larger volumes of the organ in which there was neoplasia than might be achieved using direct PDT on tumor tissue itself.

Methods. The liver of each of 20 WAG/Rij rats was implanted with a R1 rhabdomyosarcoma tumor fragment. After 2 weeks tumor growth, anesthetized rats were injected through the tail vein with a dose of a photosensitizer based on body weight: 5 mg/kg Talaporfin Sodium. A 10×1.5 mm LED light source was implanted in the liver proximal to an upstream feeding vessel. LEDs were placed at a distance ≧5 mm from the tumor to ensure that the tumor itself was outside the direct illumination effect of the PDT. After a 10-minute drug-light interval, a light dose totaling 200 J/cm was administered. Animals were sacrificed at various time points, up to 10 days post treatment, and the extent and the mechanism of cell death was evaluated histologically using TUNEL [TdT-mediated dUTP-X nick end labeling] and EM [Electron Microscopy].

Results. In those instances where tumors had been implanted downstream from the targeted vessel, cell death was consistently induced by a predominantly apoptotic mechanism. Cell death was first observed 2 days after PDT using TUNEL, and the apoptotic pathway confirmed with EM (DNA fragmentation, nuclear condensation and apoptosis bodies). In contrast, no cell death was observed in tumors located similarly close to the implanted light source, but vascularized by vessels other than the one targeted with PDT.

It was also found that when Talaporfin Sodium was dosed at 2 mg/kg or less, the PDT treatment effect was restricted to the immediate vicinity of the illumination. This dose was not sufficient to close larger blood vessels and produce the extended PDT effect of the invention.

Conclusions. These results suggest that light activated drug therapy can induce expanded volumes of cell death when specifically targeted at a tumor's feeding vessel. This finding may have important therapeutic implications for the treatment of larger tumors or tumor clusters for which surgery or other locally ablative techniques are contra-indicated. The results also show that the photosensitizer dose required for the vessel occlusion-based, extended PDT-effect is greater than that required for PDT of the immediate illumination zone. The dose required will vary depending upon the specific photosensitizer (some may cause vessel occlusion more readily than others), tissue type and species being treated.

While much of the foregoing description has focused on Talaporfin Sodium, it should be understood that the invention encompasses the use of any photosensitizer that is able to occlude new vessels effectively when excited with an appropriate wavelength of electromagnetic radiation. Thus, any of the photosensitizers listed in U.S. Pat. No. 6,800,086 at columns 6 to 20 that have this property, may be used. U.S. Pat. No. 6,800,086 is hereby incorporated by reference with respect to all named photosensitizers and families of photosensitizers. Suitable photosensitizers can include verteporfin and rostaporfin.

These and other compounds are as follows:

As used herein, the term “photosensitizer,” “photosensitizer compound,” “photosensitizing drug,” “PS,” and “photoactive agent” are used interchangeably. Any variation in meaning between these terms is not meant to depart from the meaning and scope of the claimed invention.

Examples of these and other PSs for use in the present invention include, but are not limited to, angelicins, some biological macromolecules such as lipofuscin; photosystem II reaction centers; and D1-D2-cyt b-559 photosystem II reaction centers, chalcogenapyrillium dyes, chlorins, chlorophylls, coumarins, cyanines, ceratin DNA and related compounds such as adenosine; cytosine; 2′-deoxyguanosine-5′-monophosphate; deoxyribonucleic acid; guanine; 4-thiouridine; 2′-thymidine 5′-monophosphate; thymidylyl(3′-5′)-2′-deoxyadenosine; thymidylyl(3′-5′)-2′-deoxyguanosine; thymine; and uracil, certain drugs such as adriamycin; afloqualone; amodiaquine dihydrochloride; chloroquine diphosphate; chlorpromazine hydrochloride; daunomycin; daunomycinone; 5-iminodaunomycin; doxycycline; furosemide; gilvocarcin M; gilvocarcin V; hydroxychloroquine sulfate; lumidoxycycline; mefloquine hydrochloride; mequitazine; merbromin (mercurochrome); primaquine diphosphate; quinacrine dihydrochloride; quinine sulfate; and tetracycline hydrochloride, certain flavins and related compounds such as alloxazine; flavin mononucleotide; 3-hydroxyflavone; limichrome; limiflavin; 6-methylalloxazine; 7-methylalloxazine; 8-methylalloxazine; 9-methylalloxazine; 1-methyl limichrome; methyl-2-methoxybenzoate; 5-nitrosalicyclic acid; proflavine; and riboflavin, fullerenes, metalloporphyrins, metallophthalocyanines, methylene blue derivatives, naphthalimides, naphthalocyanines, certain natural compounds such as bis(4-hydroxy-3-methoxyphenyl)-1,6-heptadiene-3,5-dione; 4-(4-hydroxy-3-methoxyphenyl)-3-buten-2-one; N-formylkynurenine; kynurenic acid; kynurenine; 3-hydroxykynurenine; DL-3-hydroxykynurenine; sanguinarine; berberine; carmane; and 5,7,9(11),22-ergostatetraene-3 β-ol, nile blue derivatives, NSAIDs (nonsteroidal anti-inflammatory drugs), perylenequinones, phenols, pheophorbides, pheophytins, photosensitizer dimers and conjugates, phthalocyanines, porphycenes, porphyrins, psoralens, purpurins, quinones, retinoids, rhodamines, thiophenes, verdins, vitamins and xanthene dyes (Redmond and Gamlin, Photochem. Photobiol., 70 (4):391-475 (1999)).

Exemplary angelicins include 3-aceto-angelicin; angelicin; 3,4′-dimethyl angelicin; 4,4′-dimethyl angelicin; 4,5′-dimethyl angelicin; 6,4′-dimethyl angelicin; 6,4-dimethyl angelicin; 4,4′,5′-trimethyl angelicin; 4,4′,5′-trimethyl-1′-thioangelicin; 4,6,4′-trimethyl-1′-thioangelicin; 4,6,4′trimethyl angelicin; 4,6,5′-trimethyl-1′-thioangelicin; 6,4,4′-trimethyl angelicin; 6,4′,5′-trimethyl angelicin; 4,6,4′,5′-tetramethyl-1′-thioangelicin; and 4,6,4′,5′-tetramethyl angelicin.

Exemplary chalcogenapyrillium dyes include pyrilium perchlorate, 4,4′-(1,3-propenyl)-bis[2,6-di(1,1-dimethylethyl)]-; pyrilium perchlorate, 2,6-bis(1,1-dimethyl-ethyl)-4-[1-[2,6-bis(1,1-dimethyl-ethyl)selenopyran-4-ylidene]-3-propenyl-; pyrilium hexofluoro phosphate, 2,6-bis-(1,1-dimethyl-ethyl)-selenopyran-4-ylidene]-3-propenyl-; pyrilium hexofluoro phosphate, 2,6-bis(1,1-dimethyl-ethyl)-selenopyran-4-ylidene]-3-propenyl-; pyrilium perchlorate, 2,6-bis(1,1-dimethyl-ethyl)-4-[1-[2,6-bis(1,1-dimethyl-ethyl)telluropyran-4-ylidene]-3-propenyl-; pyrilium hexofluoro phosphate, 2,6-bis(1,1-dimethyl-ethyl)-4-[1-[2,6-bis(1,1-dimethyl-ethyl)telluropyran-4-ylidene]-3-propenyl-; pyrilium perchlorate, 2,6-bis(1,1-dimethyl-ethyl)-4-[1-[2,6-bis(1,1-dimethyl-ethyl)thiapyran-4-ylidene]-3-propenyl]-; selenopyrilium hexofluoro phosphate, 2,6-bis(1,1-dimethyl-ethyl)-4-[1-[2,6-bis(1,1-dimethyl-ethyl)selenopyran-4-ylidene]-3-propenyl]-; selenopyrilium, 2,6-bis(1,1-dimethylethyl)-4-[1-[2,6-bis(1,1-dimethylethyl)selenopyran-4-ylidene]-3-propenyl]-; selenopyrilium percheorate, 2,6-bis(1,1-dimethyl-ethyl)-4-[1-[2,6-bis(1,1-dimethyl-ethyl)-4-[1-[2,6-bis(1,1-dimethyl-ethyl)telluropyran-4-ylidene]-3-propenyl]-; selenopyrilium hexofluoro phosphate, 2,6-bis(1,1-dimethyl-ethyl)-4-[1-[2,6-bis(1,1-dimethyl-ethyl)telluropyran-4-ylidene]-3-propenyl]-; selenopyrilium hexofluoro phosphate, 2,6-bis(1,1-dimethyl-ethyl)-4-[2-[2,6-bis(1,1-dimethyl-ethyl)selenopyran-4-ylidene]-4-(2-butenyl)]-; selenopyrilium hexofluoro phosphate, 2,6-bis(1,1-dimethyl-ethyl)-4-[2-[2,6-bis(1,1-dimethyl-ethyl)selenopyran-4-ylidene]-4-(2-pentenyl)]-; telluropyrilium tetrafluoroborate, 2,6-bis(1,1-dimethylethyl)-4-[1-[2,6-bis(1,1-dimethyl-ethyl)-telluropyran-4-ylidene]-3-propenyl]-; telluropyrilium hexofluoro phosphate, 2,6-bis(1,1-dimethyl-ethyl)-4-[1-[2,6-bis(1,1-dimethyl-ethyl)telluropyran-4-ylidene]-3-propenyl]-; telluropyrilium hexofluoro phosphate, 2,6-bis(1,1-dimethyl-ethyl)-4-[1-[2,6-bis(1,1-dimethyl-ethyl)telluropyran-4-ylidene]ethyl-; telluropyrilium hexofluoro phosphate, 2,6-bis(1,1-dimethyl-ethyl)-4-[1-[2,6-bis(1,1-dimethyl-ethyl)-telluropyran-4-ylidene]methyl-; thiopyrilium hexofluoro phosphate, 2,6-bis(1,1-dimethyl-ethyl)-4-[1-[2,6-bis(1,1-dimethyl-ethyl)thiopyran-4-ylidene]-3-propenyl]-; thiopyrilium hexofluoro phosphate, 2,6-bis(1,1-dimethyl-ethyl)-4-[1-[2,6-bis(1,1-dimethyl-ethyl)selenopyran-4-ylidene]-3-propenyl]-; and thiopyrilium hexofluoro phosphate, 2,6-bis(1,1-dimethyl-ethyl)-4-[1-[2,6-bis(1,1-dimethyl-ethyl)telluropyran-4-ylidene]-3-propenyl]-.

Exemplary chlorins dyes include 5-azachlorin dimethyl ester derivative; 5,10,15,20-tetrakis-(m-hydroxyphenyl) bacteriochlorin; benzoporphyrin derivative monoacid ring A; benzoporphyrin derivative monoacid ring-A; porphine-2,18-dipropanoic acid, 7-[2-dimethyl-amino)-2-oxoethyl]-8-ethylidene-7,8-dihydro-3,7,12,17-tetram ethyl, dimethylester; porphine-2,18-dipropanoic acid, 7-[2-dimethyl-amino)-2-oxoethyl]-8-ethylidene-8-ethyl-7,8-dihydro-3,7,12,17-tetramethyl, dimethylester Z; porphine-2,18-dipropanoic acid, 7-[2-dimethyl-amino)-2-oxoethyl]-8-ethylidene-8-ethyl-7,8-dihydro-3,7,12,17-tetramethyl, dimethylester Z ECHL; porphine-2,18-dipropanoic acid, 7-[2-dimethyl-amino)-2-oxoethyl]-8-ethylidene-8-n-heptyl-7,8-dihydro-3,7,12,17-tetramethyl, dimethylester Z; tin (II) porphine-2,18-dipropanoic acid, 7-[2-(dimethylamino-2-oxoethyl]-8-ethylidene-8-n-heptyl-7,8-dihydro-3,7,12,17-tetramethyl, dimethylester Z; chlorin e₆; chlorin e₆ dimethyl ester; chlorin e₆ k₃; chlorin e₆ monomethyl ester; chlorin e₆ Na₃; chlorin p₆; chlorin p₆-trimethylester; chlorin derivative zinc (II) porphine-2,18-dipropanoic acid, 7-[2-(dimethylamino)-2-oxoethyl]-8-ethylidene-8-n-heptyl-7,8-dihydro-3,7,12,17-tetramethyl, dimethylester Z; 13¹-deoxy-20-formyl-vic-dihydroxy-bacteriochlorin di-tert-butyl aspartate; 13¹-deoxy-20-formyl-4-keto-bacteriochlorin di-tert-butyl aspartate; di-L-aspartyl chlorin e₆; mesochlorin; 5,10,15,20-tetrakis-(m-hydroxyphenyl) chlorin; meta-(tetrahydroxyphenyl)chlorin; methyl-13¹-deoxy-20-formyl-4-keto-bacteriochlorin; mono-L-aspartyl chlorin e₆; photoprotoporphyrin IX dimethyl ester; phycocyanobilin dimethyl ester; protochlorophyllide a; tin (IV) chlorin e₆; tin chlorin e₆; tin L-aspartyl chlorin e₆; tin octaethyl-benzochlorin; tin (IV) chlorin; zinc chlorin e₆; and zinc L-aspartyl chlorin e₆.

Exemplary chlorophylls dyes include chlorophyll a; chlorophyll b; oil soluble chlorophyll; bacteriochlorophyll a; bacteriochlorophyll b; bacteriochlorophyll c; bacteriochlorophyll d; protochlorophyll; protochlorophyll a; amphiphilic chlorophyll derivative 1; and amphiphilic chlorophyll derivative 2.

Exemplary coumarins include 3-benzoyl-7-methoxycoumarin; 7-diethylamino-3-thenoylcoumarin; 5,7-dimethoxy-3-(1-naphthoyl)coumarin; 6-methylcoumarin; 2H-selenolo[3,2-g] [1] benzopyran-2-one; 2H-selenolo[3,2-g] [1] benzothiopyran-2-one; 7H-selenolo[3,2-g] [1] benzoseleno-pyran-7-one; 7H-selenopyrano[3,2-f] [1] benzofuran-7-one; 7H-selenopyrano[3,2-f] [1] benzo-thiophene-7-one; 2H-thienol[3,2-g] [1] benzopyran-2-one; 7H-thienol[3,2-g] [1] benzothiopyran-7-one; 7H-thiopyrano[3,2-f] [1] benzofuran-7-one; coal tar mixture; khellin; RG 708; RG277; and visnagin.

Exemplary cyanines include benzoselenazole dye; benzoxazole dye; 1,1′-diethyloxacarbocyanine; 1,1′-diethyloxadicarbocyanine; 1,1′-diethylthiacarbocyanine; 3,3′-dialkylthiacarbocyanines (n=2-18); 3,3′-diethylthiacarbocyanine iodide; 3,3′-dihexylselenacarbocyanine; kryptocyanine; MC540 benzoxazole derivative; MC540 quinoline derivative; merocyanine 540; and meso-ethyl, 3,3′-dihexylselenacarbocyanine.

Exemplary fullerenes include C₆₀; C₇₀; C₇₆; dihydro-fullerene; 1,9-(4-hydroxy-cyclohexano)-buckminster-fullerene; [1-methyl-succinate-4-methyl-cyclohexadiene-2,3]-buckminster-fullerene; and tetrahydro fullerene.

Exemplary metalloporphyrins include cadmium (II) chlorotexaphyrin nitrate; cadmium (II) meso-diphenyl tetrabenzoporphyrin; cadmium meso-tetra-(4-N-methylpyridyl)-porphine; cadmium (II) texaphyrin; cadmium (II) texaphyrin nitrate; cobalt meso-tetra-(4-N-methylpyridyl)-porphine; cobalt (II) meso(4-sulfonatophenyl)-porphine; copper hematoporphyrin; copper meso-tetra-(4-N-methylpyridyl)-porphine; copper (II) meso(4-sulfonatophenyl)-porphine; Europium (M) dimethyltexaphyrin dihydroxide; gallium tetraphenylporphyrin; iron meso-tetra(4-N-methylpyridyl)-porphine; lutetium (III) tetra(N-methyl-3-pyridyl)-porphyrin chloride; magnesium (II) meso-diphenyl tetrabenzoporphyrin; magnesium tetrabenzoporphyrin; magnesium tetraphenylporphyrin; magnesium (II) meso(4-sulfonatophenyl)-porphine; magnesium (II) texaphyrin hydroxide metalloporphyrin; magnesium meso-tetra-(4-N-methylpyridyl)-porphine; manganese meso-tetra-(4-N-methylpyridyl)-porphine; nickel meso-tetra(4-N-methylpyridyl)-porphine; nickel (II) meso-tetra(4-sulfonatophenyl)-porphine; palladium (II) meso-tetra-(4-N-methylpyridyl)-porphine; palladium meso-tetra-(4-N-methylpyridyl)-porphine; palladium tetraphenylporphyrin; palladium (II) meso(4-sulfonatophenyl)-porphine; platinum (II) meso(4-sulfonatophenyl)-porphine; samarium (II) dimethyltexaphyrin dihydroxide; silver (II) meso(4-sulfonatophenyl)-porphine; tin (IV) protoporphyrin; tin meso-tetra-(4-N-methylpyridyl)-porphine; tin meso-tetra(4-sulfonatophenyl)-porphine; tin (IV) tetrakis(4-sulfonatophenyl)porphyrin dichloride; zinc (II)15-aza-3,7,12,18-tetramethyl-porphyrinato-13,17-diyl-dipropionic acid-dimethylester; zinc (II) chlorotexaphyrin chloride; zinc coproporphyrin III; zinc (II) 2,11,20,30-tetra-(1,1-dimethyl-ethyl)tetranaphtho(2,3-b:2′,3′-g:2″3″-1:2′″,3′″-q)porphyrazine; zinc (II) 2-(3-pyridyloxy)benzo[b]-10,19,28-tri(1,1-dimethylethyl)trinaphtho[2′,3′-g:2″3″1::2′″,3′″-q] porphyrazine; zinc (II) 2,18-bis-(3-pyridyloxy)dibenzo[b, 1]-10,26-di(1,1-dimethyl-ethyl)dinaphtho[2′,3′-g:2′″,3′″-q]porphyrazine; zinc (II) 2,9-bis-(3-pyridyloxy)dibenzo[b,g]-17,26-di(1,1-dimethyl-ethyl)dinaphtho[2″,3″-1:2′″,3′″-q]porphyrazine; zinc (II) 2,9,16-tris-(3-pyridyloxy) tribenzo[b,g, 1]-24=(1,1-dimethyl-ethyl)naphtho[2′″,3′″-q]porphyrazine; zinc (II) 2,3-bis-(3-pyridyloxy)benzo[b]-10,19,28-tri(1,1-dimethyl-ethyl)trinaphtho[2′,3′-g:2″,3″1:2′″,3′″-q]porphyrazine; zinc (II) 2,3,18,19-tetrakis-(3-pyridyloxy)dibenzo[b, 1]-10,26-di(1,1-dimethyl-ethyl) trinaphtho[2′,3′-g:2′″,3′″-q]porphyrazine; zinc (II) 2,3,9,10-tetrakis-(3-pyridyloxy)dibenzo[b,g]-17,26-di(1,1-dimethyl-ethyl)dinaphtho[2″,3″-1:2′″,3′″-q]porphyrazine; zinc (II) 2,3,9,10,16,17-hexakis-(3-pyridyloxy)tribenzo[b,g, 1]-24-(1,1-dimethyl-ethyl)naphtho[2′″,3′″-q]porphyrazine; zinc (II) 2-(3-N-methyl)pyridyloxy)benzo[b]-10,19,28-tri(1,1-dimethyl-ethyl)trinaphtho[2′,3′-g:2″,3″1:2′″,3′″-q]porphyrazine monoiodide; zinc (II) 2,18-bis-(3-(N-methyl)pyridyloxy)dibenzo[b, 1]-10,26-di(1,1-dimethylethyl)dinaphtho[2′,3′-g:2′″,3′″-q]porphyrazine diiodide; zinc (II) 2,9-bis-(3-(N-methyl)pyridyloxy)dibenzo[b,g]-17,26-di(1,1-dimethylethyl)dinaphtho[2″,3″-1:2′″,3′″-q]porphyrazine diiodide; zinc (II) 2,9,16-tris-(3-(N-methyl-pyridyloxy)tribenzo[b,g, 1]-24-(1,1-dimethylethyl) naphtho[2′″,3′″-q]porphyrazine triiodide; zinc (II) 2,3-bis-(3-(N-methyl)pyridyloxy)benzo[b]-10,19,28-tri(1,1-dimethylethyl)trinaphtho[2′,3′-g:2″,3″-1:2′″,3′″-q]porphyrazine diiodide; zinc (II) 2,3,18,19-tetrakis-(3-(N-methyl)pyridyloxy)dibenzo[b, 1]-10,26-di(1,1-dimethyl)dinaphtho[2′,3′-g:2′″,3′″-q]porphyrazine tetraiodide; zinc (II) 2,3,9,10-tetrakis-(3-(N-methyl)pyridyloxy)dibenzo[g,g]-17,26-di(1,1-dimethylethyl)dinaphtho[2″,3″-1:2′″,3′″-q]porphyrazine tetraiodide; zinc (II) 2,3,9,10,16,17-hexakis-(3-(N-methyl)pyridyloxy)tribenzo[b,g, 1]-24-(1,1-dimethylethyl)naphtho[2′″,3′″-q]porphyrazine hexaiodide; zinc (II) mesc)-diphenyl tetrabenzoporphyrin; zinc (II) meso-triphenyl tetrabenzoporphyrin; zinc (II) meso-tetrakis(2,6-dichloro-3-sulfonatophenyl)porphyrin; zinc (II) meso-tetra-(4-N-methylpyridyl)-porphine; zinc (II) 5,10,15,20-meso-tetra(4-octyl-phenylpropynyl)-porphine; zinc porphyrin c; zinc protoporphyrin; zinc protoporphyrin IX; zinc (II) meso-triphenyl-tetrabenzoporphyrin; zinc tetrabenzoporphyrin; zinc (II) tetrabenzoporphyrin; zinc tetranaphthaloporphyrin; zinc tetraphenylporphyrin; zinc (II) 5,10,15,20-tetraphenylporphyrin; zinc (II) meso (4-sulfonatophenyl)-porphine; and zinc (II) texaphyrin chloride.

Exemplary metallophthalocyanines include aluminum mono-(6-carboxy-pentyl-amino-sulfonyl)-trisulfo-phthalocyanine; aluminum di-(6-carboxy-pentyl-amino-sulfonyl)-trisulfophthalocyanine; aluminum (III) octa-n-butoxy phthalocyanine; aluminum phthalocyanine; aluminum (III) phthalocyanine disulfonate; aluminum phthalocyanine disulfonate; aluminum phthalocyanine disulfonate (cis isomer); aluminum phthalocyanine disulfonate (clinical prep.); aluminum phthalocyanine phthalimido-methyl sulfonate; aluminum phthalocyanine sulfonate; aluminum phthalocyanine trisulfonate; aluminum (III) phthalocyanine trisulfonate; aluminum (III) phthalocyanine tetrasulfonate; aluminum phthalocyanine tetrasulfonate; chloroaluminum phthalocyanine; chloroaluminum phthalocyanine sulfonate; chloroaluminum phthalocyanine disulfonate; chloroaluminum phthalocyanine tetrasulfonate; chloroaluminum-t-butyl-phthalocyanine; cobalt phthalocyanine sulfonate; copper phthalocyanine sulfonate; copper (II) tetra-carboxy-phthalocyanine; copper (II)-phthalocyanine; copper t-butyl-phthalocyanine; copper phthalocyanine sulfonate; copper (II) tetrakis-[methylene-thio[(dimethyl-amino)methylidyne]]phthalocyanine tetrachloride; dichlorosilicon phthalocyanine; gallium (III) octa-n-butoxy phthalocyanine; gallium (II) phthalocyanine disulfonate; gallium phthalocyanine disulfonate; gallium phthalocyanine tetrasulfonate-chloride; gallium (II) phthalocyanine tetrasulfonate; gallium phthalocyanine trisulfonate-chloride; gallium (II) phthalocyanine trisulfonate; GaPcS₁ tBu₃; GaPcS₂ tBu₂; GaPcS₃ tBu₁; germanium (IV) octa-n-butoxy phthalocyanine; germanium phthalocyanine derivative; silicon phthalocyanine derivative; germanium (IV) phthalocyanine octakis-alkoxy-derivatives; iron phthalocyanine sulfonate; lead (II) 2,3,9,10,16,17,23,24-octakis(3,6-dioxaheptyloxy) phthalocyanine; magnesium t-butyl-phthalocyanine; nickel (II) 2,3,9,10,16,17,23,24-octakis(3,6-dioxaheptyloxy) phthalocyanine; palladium (II) octa-n-butoxy phthalocyanine; palladium (II) tetra(t-butyl)-phthalocyanine; (diol) (t-butyl).sub.3-phthalocyanato palladium(II); ruthenium(II) dipotassium[bis(triphenyl-phosphine-monosulphonate) phthalocyanine; silicon phthalocyanine bis(tri-n-hexyl-siloxy)-; silicon phthalocyanine bis(tri-phenyl-siloxy)-; HOSiPcOSi(CH₃)₂ (CH₂)₃ N(CH₃)₂; HOSiPcOSi(CH₃)₂ (CH₂)₃ N(CH₂ CH₃)₂; SiPc[OSi(CH₃)₂ (CH₂)₃ N(CH₃)₂]₂; SiPc[OSi(CH₃)₂ (CH₂)₃ N(CH₂ CH₃)(CH₂)₂ N(CH₃)₂]₂; tin (IV) octa-n-butoxy phthalocyanine; vanadium phthalocyanine sulfonate; zinc (II) octa-n-butoxy phthalocyanine; zinc (II) 2,3,9,10,16,17,23,24-octakis(2-ethoxy-ethoxy) phthalocyanine; zinc (II) 2,3,9,10,16,17,23,24-octakis(3,6-dioxaheptyloxy) phthalocyanine; zinc (II) 1,4,8,11,15,18,22,25-octa-n-butoxy-phthalocyanine; zn(II)-phthalocyanine-octabutoxy; zn(II)-phthalocyanine; zinc phthalocyanine; zinc (II) phthalocyanine; zinc phthalocyanine and perdeuterated zinc phthalocyanine; zinc (II) phthalocyanine disulfonate; zinc phthalocyanine disulfonate; zinc phthalocyanine sulfonate; zinc phthalocyanine tetrabromo-; zinc (II) phthalocyanine tetra-t-butyl-; zinc (II) phthalocyanine tetra-(t-butyl)-; zinc phthalocyanine tetracarboxy-; zinc phthalocyanine tetrachloro-; zinc phthalocyanine tetrahydroxyl; zinc phthalocyanine tetraiodo-; zinc ((I) tetrakis-(1,1-dimethyl-2-phthalimido)ethyl phthalocyanine; zinc (II) tetrakis-(1,1-dimethyl-2-amino)-ethyl-phthalocyanine; zinc (II) phthalocyanine tetrakis(1,1-dimethyl-2-trimethyl ammonium)ethyl tetraiodide; zinc phthalocyanine tetrasulphonate; zinc phthalocyanine tetrasulfonate; zinc (II) phthalocyanine tetrasulfonate; zinc (II) phthalocyanine trisulfonate; zinc phthalocyanine trisulfonate; zinc (II) (t-butyl).sub.3-phthalocyanine diol; zinc tetradibenzobarreleno-octabutoxy-phthalocyanine; zinc (II) 2,9,16,23,-tetrakis-(3-(N-methyl)pyridyloxy)phthalocyanine tetraiodide; and zinc (II) 2,3,9,10,16,17,23,24-octakis-(3-(N-methyl)pyridyloxy)phthalocyanine complex octaiodide; and zinc (II) 2,3,9,10,16,17,23,24-octakis-(3-pyridyloxy)phthalocyanine.

Exemplary methylene blue derivatives include 1-methyl methylene blue; 1,9-dimethyl methylene blue; methylene blue; methylene blue (16 μM); methylene blue (14 μM; methylene violet; bromomethylene violet; 4-iodomethylene violet; 1,9-dimethyl-3-dimethyl-amino-7-diethyl-amino-phenothiazine; and 1,9-dimethyl-3-diethylamino-7-dibutyl-amino-phenothiazine.

Exemplary naphthalimides blue derivatives include N,N′-bis-(hydroperoxy-2-methoxyethyl)-1,4,5,8-naphthaldiimide; N-(hydroperoxy-2-methoxyethyl)-1,8-naphthalimide; 1,8-naphthalimide; N,N′-bis(2,2-dimethoxyethyl)-1,4,5,8-naphthaldiimide; and N,N′-bis(2,2-dimethylpropyl)-1,4,5,8-naphthaldiimide.

Exemplary naphthalocyanines include aluminum t-butyl-chloronaphthalocyanine; silicon bis(dimethyloctadecylsiloxy) 2,3-naphthalocyanine; silicon bis(dimethyloctadecylsiloxy)naphthalocyanine; silicon bis(dimethylthexylsiloxy) 2,3-naphthalocyanine; silicon bis(dimethylthexylsiloxy)naphthalocyanine; silicon bis(t-butyidimethylsiloxy) 2,3-naphthalocyanine; silicon bis(tert-butyidimethylsiloxy)naphthalocyanine; silicon bis(tri-n-hexylsiloxy) 2,3-naphthalocyanine; silicon bis(tri-n-hexylsiloxy)naphthalocyanine; silicon naphthalocyanine; t-butylnaphthalocyanine; zinc (II) naphthalocyanine; zinc (II) tetraacetyl-amidonaphthalocyanine; zinc (II) tetraaminonaphthalocyanine; zinc (II) tetrabenzamidonaphthalocyanine; zinc (II) tetrahexylamidonaphthalocyanine; zinc (II) tetramethoxy-benzamidonaphthalocyanine; zinc (II) tetramethoxynaphthalocyanine; zinc naphthalocyanine tetrasulfonate; and zinc (II) tetradodecylamidonaphthalocyanine.

Exemplary nile blue derivatives include benzo[a]phenothiazinium, 5-amino-9-diethylamino-; benzo[a]phenothiazinium, 5-amino-9-diethylamino-6-iodo-; benzo[a]phenothiazinium, 5-benzylamino-9-diethylamino-; benzo[a]phenoxazinium, 5-amino-6,8-dibromo-9-ethylamino-; benzo[a]phenoxazinium, 5-amino-6,8-diiodo-9-ethylamino-; benzo[a]phenoxazinium, 5-amino-6-bromo-9-diethylamino-; benzo[a]phenoxazinium, 5-amino-9-diethylamino-(nile blue A); benzo[a]phenoxazinium, 5-amino-9-diethylamino-2,6-diiodo-; benzo[a]phenoxazinium, 5-amino-9-diethylamino-2,-iodo; benzo[a]phenoxazinium, 5-amino-9-diethylamino-6-iodo-; benzo[a]phenoxazinium, 5-benzylamino-9-diethylamino-(nile blue 2B); 5-ethylamino-9-diethylamino-benzo[a]phenoselenazinium chloride; 5-ethylamino-9-diethyl-aminobenzo[a]phenothiazinium chloride; and 5-ethylamino-9-diethyl-aminobenzo[a]phenoxazinium chloride.

Exemplary NSAIDs (nonsteroidal anti-inflammatory drugs) include benoxaprofen; carprofen; carprofen dechlorinated (2-(2-carbazolyl) propionic acid); carprofen (3-chlorocarbazole); chlorobenoxaprofen; 2,4-dichlorobenoxaprofen; cinoxacin; ciprofloxacin; decarboxy-ketoprofen; decarboxy-suprofen; decarboxy-benoxaprofen; decarboxy-tiaprofenic acid; enoxacin; fleroxacin; fleroxacin-N-oxide; flumequine; indoprofen; ketoprofen; lomelfloxacin; 2-methyl-4-oxo-2H-1,2-benzothiazine-1,1-dioxide; N-demethyl fleroxacin; nabumetone; nalidixic acid; naproxen; norfloxacin; ofloxacin; pefloxacin; pipemidic acid; piroxicam; suprofen; and tiaprofenic acid.

Exemplary perylenequinones include hypericins such as hypericin; hypericin monobasic sodium salt; di-aluminum hypericin; di-copper hypericin; gadolinium hypericin; terbium hypericin, hypocrellins such as acetoxy hypocrellin A; acetoxy hypocrellin B; acetoxy iso-hypocrellin A; acetoxy iso-hypocrellin B; 3,10)-bis[2-(2-aminoethylamino)ethanol] hypocrellin B; 3,10-bis[2-(2-aminoethoxy)ethanol] hypocrellin B; 3,10-bis[4-(2-aminoethyl)morpholine] hypocrellin B; n-butylaminated hypocrellin B; 3,10-bis(butylamine) hypocrellin B; 4,9-bis(butylamine) hypocrellin B; carboxylic acid hypocrellin B; cystamine-hypocrellin B; 5-chloro hypocrellin A or 8-chloro hypocrellin A; 5-chloro hypocrellin B or 8-chloro hypocrellin B; 8-chloro hypocrellin B; 8-chloro hypocrellin A or 5-chloro hypocrellin A; 8-chloro hypocrellin B or 5-chloro hypocrellin B; deacetylated aldehyde hypocrellin B; deacetylated hypocrellin B; deacetylated hypocrellin A; deacylated, aldehyde hypocrellin B; demethylated hypocrellin B; 5,8-dibromo hypocrellin A; 5,8-dibromo hypocrellin B; 5,8-dibromo iso-hypocrellin B; 5,8-dibromo[1,12-CBr═CMeCBr(COMe)] hypocrellinB; 5,8-dibromo[1,12-CHBrC(═CH₂)CBr(COMe)] hypocrellin B; 5,8-dibromo[1-CH.sub.2 CO Me, 12-COCOCH.sub.2 Br—] hypocrellin B; 5,8-dichloro hypocrellin A; 5,8-dichloro hypocrellin B; 5,8-dichlorodeacytylated hypocrellin B; 5,8-diiodo hypocrellin A; 5,8-diiodo hypocrellin B; 5,8-diiodo[1,12-CH═CMeCH(COCH₂ I₂)—] hypocrellin B; 5,8-diiodo[1,12-CH₂ C(CH₂ I)═C(COMe)—] hypocrellin B; 2-(N,N-diethylamino) ethylaminated hypocrellin B; 3,10-bis[2-(N,N-diethylamino)-ethylamine]hypocrellin B; 4,9-bis[2-(N,N-diethyl-amino)-ethylamine] iso-hypocrellin B; dihydro-1,4-thiazine carboxylic acid hypocrellin B; dihydro-1,4-thiazine hypocrellin B; 2-(N,N-dimethylamino) propylamine hypocrellin B; dimethyl-1,3,5,8,10,12-hexamethoxy-4,9-perylenequinone-6,7-diacetate; dimethyl-5,8-dihydroxy-1,3,10,13-tetramethoxy-4,9-perylenequinone-6,7-diacetate; 2,11-dione hypocrellin A; ethanolamine hypocrellin B; ethanolamine iso-hypocrellin B; ethylenediamine hypocrellin B; 11-hydroxy hypocrellin B or 2-hydroxy hypocrellin B; hypocrellin A; hypocrellin B; 5-iodo[1,12-CH₂ C(CH₂ I)═C(COMe)—] hypocrellin B; 8-iodo[1,12-CH₂ C(CH₂ I)═C(COMe)—] hypocrellin B; 9-methylamino iso-hypocrellin B; 3,10-bis[2-(N,N-methylamino)propylamine]hypocrellin B; 4,9-bis(methylamine iso-hypocrellin B; 14-methylamine iso-hypocrellin B; 4-methylamine iso-hypocrellin B; methoxy hypocrellin A; methoxy hypocrellin B; methoxy iso-hypocrellin A; methoxy iso-hypocrellin B; methylamine hypocrellin B; 2-morpholino ethylaminated hypocrellin B; pentaacetoxy hypocrellin A; PQP derivative; tetraacetoxy hypocrellin B; 5,8,15-tribromo hypocrellin B; calphostin C, Cercosporins such as acetoxy cercosporin; acetoxy iso-cercosporin; aminocercosporin; cercosporin; cercosporin+iso-cercosporin (1/1 molar); diaminocercosporin; dimethylcercosporin; 5,8-dithiophenol cercosporin; iso-cercosporin; methoxycercosporin; methoxy iso-cercosporin; methylcercosporin; noranhydrocercosporin; elsinochrome A; elsinochrome B; phleichrome; and rubellin A.

Exemplary phenols include 2-benzylphenol; 2,2′-dihydroxybiphenyl; 2,5-dihydroxybiphenyl; 2-hydroxybiphenyl; 2-methoxybiphenyl; and 4-hydroxybiphenyl.

Exemplary pheophorbides include pheophorbide a; methyl 13.sup.1-deoxy-20-formyl-7,8-vic-dihydro-bacterio-meso-pheophorbide a; methyl-2-(1-dodecyloxyethyl)-2-devinyl-pyropheophorbide a; methyl-2-(1-heptyl-oxyethyl)-2-devinyl-pyropheophorbide a; methyl-2-(1-hexyl-oxyethyl)-2-devinyl-pyropheophorbide a; methyl-2-(1-methoxy-ethyl)-2-devinyl-pyropheophorbide a; methyl-2-(1-pentyl-oxyethyl)-2-devinyl-pyropheophorbide a; magnesium methyl bacteriopheophorbide d; methyl-bacteriopheophorbide d; and pheophorbide.

Exemplary pheophytins include bacteriopheophytin a; bacteriopheophytin b; bacteriopheophytin c; bacteriopheophytin d; 10-hydroxy pheophytin a; pheophytin; pheophytin a; and protopheophytin.

Exemplary photosensitizer dimers and conjugates include aluminum mono-(6-carboxy-pentyl-amino-sulfonyl)-trisulfophthalocyanine bovine serum albumin conjugate; dihematoporphyrin ether (ester); dihematoporphyrin ether; dihematoporphyrin ether (ester)-chlorin; hematoporphyrin-chlorin ester; hematoporphyrin-low density lipoprotein conjugate; hematoporphyrin-high density lipoprotein conjugate; porphine-2,7,18-tripropanoic acid, 13,13′-(1,3-propanediyl)bis[3,8,12,17-tetramethyl]-; porphine-2,7,18-tripropanoic acid, 13,13′-(1,11-undecanediyl)bis[3,8,12,17-tetramethyl]-; porphine-2,7,18-tripropanoic acid, 13,13′-(1,6-hexanediyl)bis[3,8,12,17-tetramethyl]-; SnCe6-MAb conjugate 1.7:1; SnCe6-MAb conjugate 1.7:1; SnCe6-MAb conjugate 6.8:1; SnCe6-MAb conjugate 11.2:1; SnCe6-MAb conjugate 18.9:1; SnCe6-dextran conjugate 0.9:1; SnCe6-dextran conjugate 3.5:1; SnCe6-dextran conjugate 5.5:1; SnCe6-dextran conjugate 9.9:1; α-terthienyl-bovine serum albumin conjugate (12:1); α-terthienyl-bovine serum albumin conjugate (4:1); and tetraphenylporphine linked to 7-chloroquinoline.

Exemplary phthalocyanines include (diol) (t-butyl)₃-phthalocyanine; (t-butyl)₄-phthalocyanine; cis-octabutoxy-dibenzo-dinaphtho-porphyrazine; trans-octabutoxy-dibenzo-dinaphtho-porphyrazine; 2,3,9,10,16,17,23,24-octakis2-ethoxyethoxy) phthalocyanine; 2,3,9,10,16,17,23,24-octakis(3,6-dioxaheptyloxy) phthalocyanine; octa-n-butoxy phthalocyanine; phthalocyanine; phthalocyanine sulfonate; phthalocyanine tetrasulphonate; phthalocyanine tetrasulfonate; t-butyl-phthalocyanine; tetra-t-butyl phthalocyanine; and tetradibenzobarreleno-octabutoxy-phthalocyanine.

Exemplary porphycenes include 2,3-(2³-carboxy-2⁴-methoxycarbonyl benzo)-7,12,17-tris(2-methoxyethyl) porphycene; 2-(2-hydroxyethyl)-7,12,17-tri(2-methoxyethyl) porphycene; 2-(2-hydroxyethyl)-7,12,17-tri-n-propyl-porphycene; 2-(2-methoxyethyl)-7,12,17-tri-n-propyl-porphycene; 2,7,12,17-tetrakis(2-methoxyethyl) porphycene; 2,7,12,17-tetrakis(2-methoxyethyl)-9-hydroxy-porphycene; 2,7,12,17-tetrakis(2-methoxyethyl)-9-methoxy-porphycene; 2,7,12,17-tetrakis(2-methoxyethyl)-9-n-hexyloxy-porphycene; 2,7,12,17-tetrakis(2-methoxyethyl)-9-acetoxy-porphycene; 2,7,12,17-tetrakis(2-methoxyethyl)-9-caproyloxy-porphycene; 2,7,12,17-tetrakis(2-methoxyethyl)-9-pelargonyloxy-porphycene; 2,7,12,17-tetrakis(2-methoxyethyl)-9-stearoyloxy-porphycene; 2,7,12,17-tetrakis(2-methoxyethyl)-9-(N-t-butoxycarbonylglycinoxy) porphycene; 2,7,12,17-tetrakis(2-methoxyethyl)-9-[4-((β-apo-7-carotenyl)benzoylox yl-porphycene; 2,7,12,17-tetrakis(2-methoxyethyl)-9-amino-porphycene; 2,7,12,17-tetrakis(2-methoxyethyl)-9-acetamido-porphycene; 2,7,12,17-tetrakis(2-methoxyethyl)-9-glutaramido-porphycene; 2,7,12,17-tetrakis(2-methoxyethyl)-9-(methyl-glutaramido)-porphycene; 2,7,12,17-tetrakis(2-methoxyethyl)-9-(glutarimido)-porphycene; 2,7,12,17-tetrakis(2-methoxyethyl)-3-(N,N-dimethylaminomethyl)-porphycene; 2,7,12,17-tetrakis(2-methoxyethyl)-3-(N,N-dimethylaminomethyl)-porphycene hydrochloride; 2,7,12,17-tetrakis(2-ethoxyethyl)-porphycene; 2,7,12,17-tetra-n-propyl-porphycene; 2,7,12,17-tetra-n-propyl-9-hydroxy-porphycene; 2,7,12,17-tetra-n-propyl-9-methoxy-porphycene; 2,7,12,17-tetra-n-propyl-9-acetoxy porphycene; 2,7,12,17-tetra-n-propyl-9-(t-butyl glutaroxy)-porphycene; 2,7,12,17-tetra-n-propyl-9-(N-t-butoxycarbonylglycinoxy)-porphycene; 2,7,12,17-tetra-n-propyl-9-(4-N-t-butoxy-carbonyl-butyroxy)-porphycene; 2,7,12,17-tetra-n-propyl-9-amino-porphycene; 2,7,12,17-tetra-n-propyl-9-acetamido-porphycene; 2,7,12,17-tetra-n-propyl-9-glutaramido-porphycene; 2,7,12,17-tetra-n-propyl-9-(methyl glutaramido)-porphycene; 2,7,12,17-tetra-n-propyl-3-(N,N-dimethylaminomethyl) porphycene; 2,7,12,17-tetra-n-propyl-9,10-benzo porphycene; 2,7,12,17-tetra-n-propyl-9-p-benzoyl carboxy-porphycene; 2,7,12,17-tetra-n-propyl-porphycene; 2,7,12,17-tetra-t-butyl-3,6; 13,16-dibenzo-porphycene; 2,7-bis(2-hydroxyethyl)-12,17-di-n-propyl-porphycene; 2,7-bis(2-methoxyethyl)-12,17-di-n-propyl-porphycene; and porphycene.

Exemplary porphyrins include 5-azaprotoporphyrin dimethylester; bis-porphyrin; coproporphyrin III; coproporphyrin III tetramethylester; deuteroporphyrin; deuteroporphyrin IX dimethylester; diformyldeuteroporphyrin IX dimethylester; dodecaphenylporphyrin; hematoporphyrin; hematoporphyrin (8 μM); hematoporphyrin (400 μM); hematoporphyrin (3 μM); hematoporphyrin (18 μM); hematoporphyrin (30 μM); hematoporphyrin (67 μM); hematoporphyrin (150 μM); hematoporphyrin IX; hematoporphyrin monomer; hematoporphyrin dimer; hematoporphyrin derivative; hematoporphyrin derivative (6 μM); hematoporphyrin derivative (200 μM); hematoporphyrin derivative A (20 μM); hematoporphyrin IX dihydrochloride; hematoporphyrin dihydrochloride; hematoporphyrin IX dimethylester; haematoporphyrin IX dimethylester; mesoporphyrin dimethylester; mesoporphyrin IX dimethylester; monoformyl-monovinyl-deuteroporphyrin IX dimethylester; monohydroxyethylvinyl deuteroporphyrin; 5,10,15,20-tetra(o-hydroxyphenyl) porphyrin; 5,10,15,20-tetra(m-hydroxyphenyl) porphyrin; 5,10,15,20-tetrakis-(m-hydroxyphenyl) porphyrin; 5,10,15,20-tetra(p-hydroxyphenyl) porphyrin; 5,10,15,20-tetrakis (3-methoxyphenyl) porphyrin; 5,10,15,20-tetrakis (3,4-dimethoxyphenyl) porphyrin; 5,10,15,20-tetrakis (3,5-dimethoxyphenyl) porphyrin; 5,10,15,20-tetrakis (3,4,5-trimethoxyphenyl) porphyrin; 2,3,7,8,12,13,17,18-octaethyl-5,10,15,20-tetraphenylporphyrin; Photofrin.®.; Photofrin.®. II; porphyrin c; protoporphyrin; protoporphyrin IX; protoporphyrin dimethylester; protoporphyrin IX dimethylester; protoporphyrin propylaminoethylformamide iodide; protoporphyrin N,N-dimethylaminopropylformamide; protoporphyrin propylaminopropylformamide iodide; protoporphyrin butylformamide; protoporphyrin N,N-dimethylamino-formamide; protoporphyrin formamide; sapphyrin 13,12,13,22-tetraethyl-2,7,18,23 tetramethyl sapphyrin-8,17-dipropanol; sapphyrin 23,12,13,22-tetraethyl-2,7,18,23 tetramethyl sapphyrin-8-monoglycoside; sapphyrin 3; meso-tetra-(4-N-carboxyphenyl)-porphine; tetra-(3-methoxyphenyl)-porphine; tetra-(3-methoxy-2,4-difluorophenyl)-porphine; 5,10,15,20-tetrakis(4-N-methylpyridyl) porphine; meso-tetra -(4-N-methylpyridyl)-porphine tetrachloride; meso-tetra(4-N-methylpyridyl)-porphine; meso-tetra-(3-N-methylpyridyl)-porphine; meso-tetra-(2-N-methylpyridyl)-porphine; tetra(4-N,N,N-trimethylanilinium) porphine; meso-tetra-(4-N,N,N″-trimethylamino-phenyl) porphine tetrachloride; tetranaphthaloporphyrin; 5,10,15,20-tetraphenylporphyrin; tetraphenylporphyrin; meso-tetra-(4-N-sulfonatophenyl)-porphine; tetraphenylporphine tetrasulfonate; meso-tetra(4-sulfonatophenyl)porphine; tetra(4-sulfonatophenyl)porphine; tetraphenylporphyrin sulfonate; meso-tetra(4-sulfonatophenyl)porphine; tetrakis (4-sulfonatophenyl)porphyrin; meso-tetra(4-sulfonatophenyl)porphine; meso(4-sulfonatophenyl)porphine; meso-tetra(4-sulfonatophenyl)porphine; tetrakis(4-sulfonatophenyl)porphyrin; meso-tetra(4-N-trimethylanilinium)-porphine; uroporphyrin; uroporphyrin 1(17 μM); uroporphyrin IX; and uroporphyrin I (18 μM).

Exemplary psoralens include psoralen; 5-methoxypsoralen; 8-methoxypsoralen; 5,8-dimethoxypsoralen; 3-carbethoxypsoralen; 3-carbethoxy-pseudopsoralen; 8-hydroxypsoralen; pseudopsoralen; 4,5′,8-trimethylpsoralen; allopsoralen; 3-aceto-allopsoralen; 4,7-dimethyl-allopsoralen; 4,7,4′-trimethyl-allopsoralen; 4,7,5′-trimethyl-allopsoralen; isopseudopsoralen; 3-acetoisopseudopsoralen; 4,5′-dimethyl-isopseudopsoralen; 5′,7-dimethyl-isopseudopsoralen; pseudoisopsoralen; 3-acetopseudoisopsoralen; 3/4′,5′-trimethyl-aza-psoralen; 4,4′,8-trimethyl-5′-amino-methylpsoralen; 4,4′,8-trimethyl-phthalamyl-psoralen; 4,5′,8-trimethyl-4′-aminomethyl psoralen; 4,5′,8-trimethyl-bromopsoralen; 5-nitro-8-methoxy-psoralen; 5′-acetyl-4,8-dimethyl-psoralen; 5′-aceto-8-methyl-psoralen; and 5′-aceto-4,8-dimethyl-psoralen.

Exemplary purpurins include octaethylpurpurin; octaethylpurpurin zinc; oxidized octaethylpurpurin; reduced octaethylpurpurin; reduced octaethylpurpurin tin; purpurin 18; purpurin-18; purpurin-18-methyl ester; purpurin; tin ethyl etiopurpurin I; Zn(II) aetio-purpurin ethyl ester; and zinc etiopurpurin.

Exemplary quinones include 1-amino-4,5-dimethoxy anthraquinone; 1,5-diamino-4,8-dimethoxy anthraquinone; 1,8-diamino-4,5-dimethoxy anthraquinone; 2,5-diamino-1,8-dihydroxy anthraquinone; 2,7-diamino-1,8-dihydroxy anthraquinone; 4,5-diamino-1,8-dihydroxy anthraquinone; mono-methylated 4,5- or 2,7-diamino-1,8-dihydroxy anthraquinone; anthralin (keto form); anthralin; anthralin anion; 1,8-dihydroxy anthraquinone; 1,8-dihydroxy anthraquinone (Chrysazin); 1,2-dihydroxy anthraquinone; 1,2-dihydroxy anthraquinone (Alizarin); 1,4-dihydroxy anthraquinone (Quinizarin); 2,6-dihydroxy anthraquinone; 2,6-dihydroxy anthraquinone (Anthraflavin); 1-hydroxy anthraquinone (Erythroxy-anthraquinone); 2-hydroxy-anthraquinone; 1,2,5,8-tetra-hydroxy anthraquinone (Quinalizarin); 3-methyl-1, 6, 8-trihydroxy anthraquinone (Emodin); anthraquinone; anthraquinone-2-sulfonic acid; benzoquinone; tetramethyl benzoquinone; hydroquinone; chlorohydroquinone; resorcinol; and 4-chlororesorcinol.

Exemplary retinoids include all-trans retinal; C₁₇ aldehyde; C₂₂ aldehyde; 11-cis retinal; 13-cis retinal; retinal; and retinal palmitate.

Exemplary rhodamines include 4,5-dibromo-rhodamine methyl ester; 4,5-dibromo-rhodamine n-butyl ester; rhodamine 101 methyl ester; rhodamine 123; rhodamine 6G; rhodamine 6G hexyl ester; tetrabromo-rhodamine 123; and tetramethyl-rhodamine ethyl ester.

Exemplary thiophenes include terthiophenes such as 2,2′:5′,2″-terthiophene; 2,2′:5′,2″-terthiophene-5-carboxamide; 2,2′:5′,2″-terthiophene-5-carboxylic acid; 2,2′:5′,2″-terthiophene-5-L-serine ethyl ester; 2,2′:5′,2″-terthiophene-5-N-isopropynyl-formamide; 5-acetoxymethyl-2,2′:5′,2″-terthiophene; 5-benzyl-2,2′:5′,2″-terthiophene-sulphide; 5-benzyl-2,2′:5′,2″-terthiophene-sulfoxide; 5-benzyl-2,2′:5′,2″-terthiophene-sulphone; 5-bromo-2,2′:5′,2″-terthiophene; 5-(butynyl-3′″-hydroxy)-2,2′:5′,2″-terthiophene; 5-carboxyl-5″-trimethylsilyl-2,2′:5′,2″-terthiophene; 5-cyano-2,2′:5′,2″-terthiophene; 5,5″-dibromo-2,2′:5′,2″-terthiophene; 5-(1′″,1′″-dibromoethenyl)-2,2′:5′,2″-terthiophene; 5,5″-dicyano-2,2′:5′,2″-terthiophene; 5,5″-diformyl-2,2′:5′,2″-terthiophene; 5-difluoromethyl-2,2′:5′,2″-terthiophene; 5,5″-diiodo-2,2′:5′,2″-terthiophene; 3,3″-dimethyl-2,2′:5′,2″-terthiophene; 5,5″-dimethyl-2,2′:5′,2″-terthiophene; 5-(3′″,3′″-dimethylacryloyloxymethyl)-2,2′:5′,2″-terthiophene; 5,5″-di-(t-butyl)-2,2′:5′,2″-terthiophene; 5,5″-dithiomethyl-2,2′:5′,2″-terthiophene; 3′-ethoxy-2,2′:5′,2″-terthiophene; ethyl 2,2′:5′,2″-terthiophene-5-carboxylic acid; 5-formyl-2,2′:5′,2″-terthiophene; 5-hydroxyethyl-2,2′:5′,2″-terthiophene; 5-hydroxymethyl-2,2′:5′,2″-terthiophene; 5-iodo-2,2′:5′,2″-terthiophene; 5-methoxy-2,2′:5′,2″-terthiophene; 3′-methoxy-2,2′:5′,2″-terthiophene; 5-methyl-2,2′:5′,2″-terthiophene; 5-(3′″-methyl-2′″-butenyl)-2,2′:5′,2″-terthiophene; methyl 2,2′:5′,2″-terthiophene-5-[3′″-acrylate]; methyl 2,2′:5′,2″-terthiophene-5-(3′″-propionate); N-allyl-2,2′:5′,2″-terthiophene-5-sulphonamide; N-benzyl-2,2′:5′,2″-terthiophene-5-sulphonamide; N-butyl-2,2′:5′,2″-terthiophene-5-sulphonamide; N,N-diethyl-2,2′:5′,2″-terthiophene-5-sulphonamide; 3,3′,4′,3″-tetramethyl-2,2′:5′,2″-terthiophene; 5-t-butyl-5″-trimethylsilyl-2,2′:5′,2″-terthiophene; 3′-thiomethyl-2,2′:5′,2″-terthiophene; 5-thiomethyl-2,2′:5′,2″-terthiophene; 5-trimethylsilyl-2,2′:5′,2″-terthiophene, bithiophenes such as 2,2′-bithiophene; 5-cyano-2,2′-bithiophene; 5-formyl-2,2′-bithiophene; 5-phenyl-2,2′-bithiophene; 5-(propynyl)-2,2′-bithiophene; 5-(hexynyl)-2,2′-bithiophene; 5-(octynyl)-2,2′-bithiophene; 5-(butynyl-4″-hydroxy)-2,2′-bithiophene; 5-(pentynyl-5″-hydroxy)-2,2′-bithiophene; 5-(3″,4″-dihydroxybutynyl)-2,2′-bithiophene derivative; 5-(ethoxybutynyl)-2,2′-bithiophene derivative, and misclaneous thiophenes such as 2,5-diphenylthiophene; 2,5-di(2-thienyl)furan; pyridine, 2,6-bis(2-thienyl)-; pyridine, 2,6-bis(thienyl)-; thiophene, 2-(1-naphthalenyl)-; thiophene, 2-(2-naphthalenyl)-; thiophene, 2,2′-(1,2-phenylene)bis-; thiophene, 2,2′-(1,3-phenylene)bis-; thiophene, 2,2′-(1,4-phenylene)bis-; 2,2′:5′,2″:5″,2′″-quaterthiophene; α-quaterthienyl; α-tetrathiophene; α-pentathiophene; α-hexathiophene; and α-heptathiophene.

Exemplary verdins include copro (II) verdin trimethyl ester; deuteroverdin methyl ester; mesoverdin methyl ester; and zinc methyl pyroverdin.

Exemplary vitamins include ergosterol (provitamin D2); hexamethyl-Co a Co b-dicyano-7-de(carboxymethyl)-7,8-didehydro-cobyrinate (Pyrocobester); pyrocobester; and vitamin D3.

Exemplary xanthene dyes include Eosin B (4′,5′-dibromo, 2′,7′-dinitro-fluorescein, dianion); eosin Y; eosin Y (2′,4′,5′,7′-tetrabromo-fluorescein, dianion); eosin (2′,4′,5′,7′-tetrabromo-fluorescein, dianion); eosin (2′,4′,5′,7′-tetrabromo-fluorescein, dianion) methyl ester; eosin (2′,4′,5′,7′-tetrabromo-fluorescein, monoanion) p-isopropylbenzyl ester; eosin derivative (2′,7′-dibromo-fluorescein, dianion); eosin derivative (4′,5′-dibromo-fluorescein, dianion); eosin derivative (2′,7′-dichloro-fluorescein, dianion); eosin derivative (4′,5′-dichloro-fluorescein, dianion); eosin derivative (2′,7′-diiodo-fluorescein, dianion); eosin derivative (4′,5′-diiodo-fluorescein, dianion); eosin derivative (tribromo-fluorescein, dianion); eosin derivative (2′,4′,5′,7′-tetrachloro-fluorescein, dianion); eosin; eosin dicetylpyridinium chloride ion pair; erythrosin B (2′,4′,5′,7′-tetraiodo-fluorescein, dianion); erythrosin; erythrosin dianion; erythrosin B; fluorescein; fluorescein dianion; phloxin B (2′,4′,5′,7′-tetrabromo-3,4,5,6-tetrachloro-fluorescein, dianion); phloxin B (tetrachloro-tetrabromo-fluorescein); phloxine B; rose bengal (3,4,5,6-tetrachloro-2′,4′,5′,7′-tetraiodofluorescein, dianion); rose bengal; rose bengal dianion; rose bengal O-methyl-methylester; rose bengal 6′-O-acetyl ethyl ester; rose bengal benzyl ester diphenyl-diiodonium salt; rose bengal benzyl ester triethylammonium salt; rose bengal benzyl ester, 2,4,6,-triphenylpyrilium salt; rose bengal benzyl ester, benzyltriphenyl-phosphonium salt; rose bengal benzyl ester, benzyltriphenyl phosphonium salt; rose bengal benzyl ester, diphenyl-iodonium salt; rose bengal benzyl ester, diphenyl-methylsulfonium salt; rose bengal benzyl ester, diphenyl-methyl-sulfonium salt; rose bengal benzyl ester, triethyl-ammonium salt; rose bengal benzyl ester, triphenyl pyrilium; rose bengal bis (triethyl-ammonium) salt) (3,4,5,6-tetrachloro-2′,4′,5′,7′-tetraiodofluorescein, bis (triethyl-ammonium salt); rose bengal bis (triethyl-ammonium) salt; rose bengal bis(benzyl-triphenyl-phosphonium) salt (3,4,5,6-tetrachloro-2′,4′,5′,7′-tetraiodofluorescein, bis(benzyl-triphenyl-phosphonium) salt); rose bengal bis(diphenyl-iodonium) salt (3,4,5,6-tetrachloro-2′,4′,5′,7′-tetraiodofluorescein, bis(diphenyl-iodonium) salt); rose bengal di-cetyl-pyridinium chloride ion pair; rose bengal ethyl ester triethyl ammonium salt; rose bengal ethyl ester triethyl ammonium salt; rose bengal ethyl ester; rose bengal methyl ester; rose bengal octyl ester tri-n-butyl-ammonium salt RB; rose bengal, 6′-O-acetyl-, and ethyl ester.

Particularly preferred PSs are the green porphyrins, such as BPD-DA, -DB, -MA, and -MB, and in particular BPD-MA, EA6, and B3. These compounds are porphyrin derivatives obtained by reacting a porphyrin nucleus with an alkyne in a Diels-Alder type reaction to obtain a monohydrobenzoporphyrin, and they are described in detail in the issued U.S. Pat. No. 5,171,749, which is hereby incorporated in its entirety by reference. Other photosensitizers that may be used in the present invention include those described in U.S. Pat. Nos. 5,308,608, 6,093,739, 5,703,230, 5,831,088, 5,726,304, and 5,405,957. Of course, combinations of photosensitizers may also be used. It is preferred that the absorption spectrum of the photosensitizer be in the visible range, typically between 350 nm and 1200 nm, more preferably between 400-900 nm, and even more preferably between 600-900 nm.

BPD-MA is described, for example, in U.S. Pat. No. 5,171,749; EA6 and B3 are described in U.S. Pat. Nos. 5,929,105 and 5,880,145, respectively, all of which are incorporated herein by reference. Preferred green porphyrins have the basic structure:

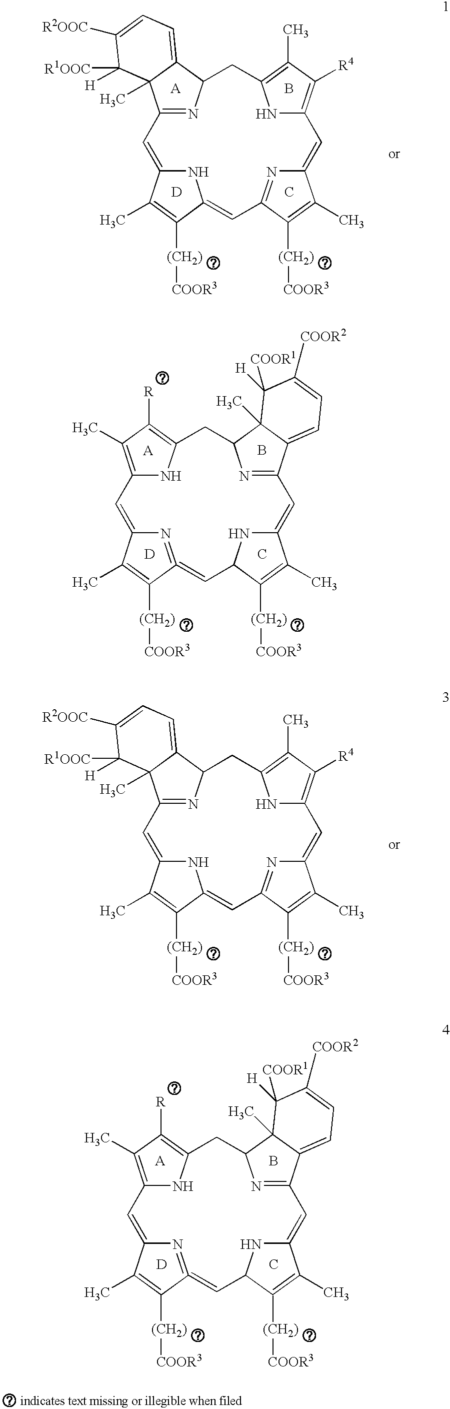

where R⁴ is vinyl or 1-hydroxyethyl and R¹, R², and R³ are H or alkyl or substituted alkyl.

BPD-MA has the structure shown in formula 1 wherein R¹ and R² are methyl, R⁴ is vinyl and one of R³ is H and the other is methyl. EA6 is of formula 2 wherein R¹ and R² are methyl and both R³ are 2-hydroxyethyl (i.e., the ethylene glycol esters). B3 is of formula 2 wherein R¹ is methyl, R² is H, and both R³ are methyl. In both EA6 and B3, R⁴ is also vinyl.

The representations of BPD-MAC and BPD-MAD, which are the components of Verteporfin, as well as illustrations of A and B ring forms of EA6 and B3, are as follows:

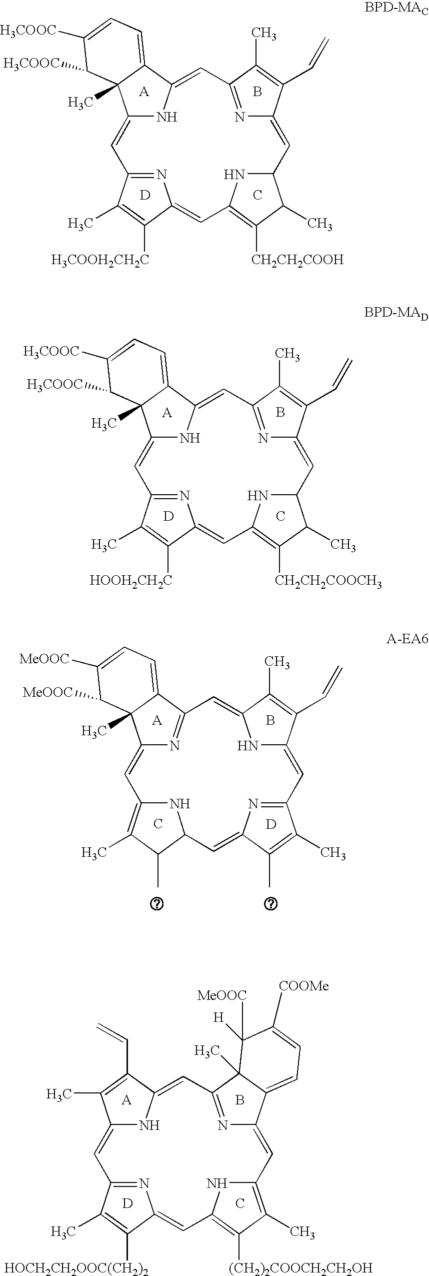

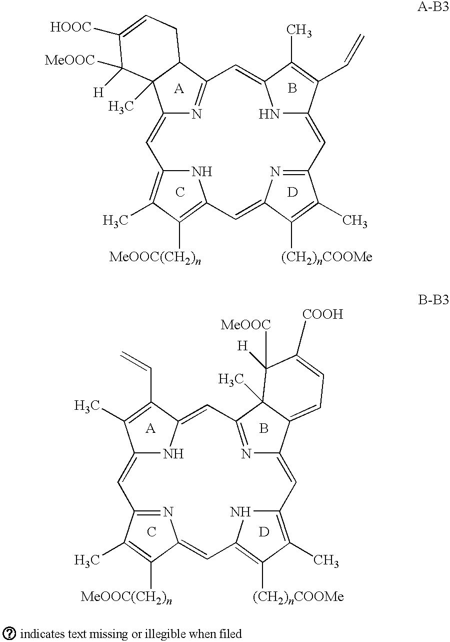

Related compounds of formulas 3 and 4 are also useful; in general, R⁴ will be vinyl or 1-hydroxyethyl and R¹, R², and R³ are H or alkyl or substituted alkyl.

The invention has been described with reference to preferred embodiments and examples, but the invention is not limited to these. Instead, the invention embodies all that is described and claimed (here below) and any changes and modifications that a person of ordinary skill in the art will readily perceive, as well as any and all equivalents to the claimed invention.

1. A method of treating a subject having a condition susceptible to treatment through occlusion of blood vessels, the treatment comprising:
a) identifying at least one blood vessel to be occluded;
b) selecting a photosensitizer that occludes blood vessels upon excitation with an appropriate wavelength of radiation;
c) delivering to the subject a dose of the photosensitizer, sufficient to cause vessel occlusion after excitation; and
d) exciting photosensitizer in the vicinity of the blood vessel with an appropriate light wavelength for a sufficient period of time to cause occlusion of the blood vessel.

2. The method of claim 1, wherein the identifying comprises locating a position of the at least one vessel.

3. The method of claim 1, wherein the selecting comprises selecting any one of the photosensitizers from the group consisting of Talaporfin Sodium, verteporfin, and rostaporfin.

4. The method of claim 3, wherein the photosensitizer is Talaporfin Sodium and the wavelength of the exciting is about 664 nm.

5. The method of claim 1, wherein the blood vessel supplies nutrients to tissue to be treated.

6. The method of claim 1, wherein the at least one blood vessel supplies nutrient to one tumor.

7. The method of claim 1, wherein the identified at least one vessel is a supplier of blood to multiple tumors.

8. A method of treating diseased tissue in a subject, the treatment comprising:
a) identifying diseased or unwanted tissue and at least one blood vessel supplying the tissue;
b) selecting a photosensitizer that occludes blood vessels upon excitation with an appropriate wavelength of radiation;
c) delivering to the subject a therapeutically effective dose of the photosensitizer; and
d) exciting the photosensitizer in the vicinity of the blood vessel for a sufficient period of time to cause occlusion of the blood vessel;
whereby occlusion of the blood vessel results in beneficial treatment of the diseased or unwanted tissue and cell death of other tissue served by the same blood vessel.

9. The method of claim 8, wherein the identifying comprises locating a position of the at least one vessel.

10. The method of claim 8, wherein the selecting comprises selecting any one of the photosensitizers from the group consisting of Talaporfin Sodium, verteporfin, and rostaporfin.

11. The method of claim 10, wherein the photosensitizer is Talaporfin Sodium and the wavelength of the exciting is about 664 nm.

12. The method of claim 8, wherein the blood vessel sup plies nutrients to tissue to be treated.

13. The method of claim 8, wherein the at least one blood vessel supplies nutrient to diseased tissue comprising at least one tumor.

14. The method of claim 8, wherein the identified at least one vessel is a supplier of blood to an organ comprising multiple tumors.

15. A method of treating an extended area of a body organ of a subject using photodynamic therapy, the organ containing a plurality of tumors, the method comprising:
a) identifying the extended area of the organ containing a plurality of tumors, and at least one major blood vessel supplying the extended area and tumors therein;
b) selecting a photosensitizer that occludes blood vessels upon excitation with an appropriate wavelength of radiation;
c) delivering to the subject a therapeutically effective dose of the photosensitizer; and
d) exciting the photosensitizer around the identified major blood vessel for a sufficient period of time to cause occlusion of the identified blood vessel.

16. The method of claim 15, wherein the identifying comprises locating a position of the at least one major vessel.

17. The method of claim 15, wherein the selecting comprises selecting any one of the photosensitizers from the group consisting of Talaporfin Sodium, verteporfin, and rostaporfin.

18. The method of claim 15, wherein the photosensitizer is Talaporfin Sodium and the wavelength of the exciting is about 664 nm.

19. The method of claim 15, wherein occlusion of the major blood vessel results in cell death of tumor cells and also of healthy tissue within the extended area.
